(19) United States
(12) Patent Application Publication
(10) Pub. No.: US 2024/0057876 A1
(43) Pub. Date: Feb. 22, 2024

(54) MEDICAL MONITORING AND REPORTING DEVICE

(71) Applicant: Sheikh K. Jasimuddin, Orlando, FL (US)

(72) Inventor: Sheikh K. Jasimuddin, Orlando, FL (US)

(21) Appl. No.: 18/384,317

(22) Filed: Oct. 26, 2023

Related U.S. Application Data
(63) Continuation-in-part of application No. 17/128,574, filed on Dec. 21, 2020.
(60) Provisional application No. 63/419,483, filed on Oct. 26, 2022.
(60) Provisional application No. 63/545,321, filed on Oct. 23, 2023.

(51) Int. Cl. A61B 5/0205; A61B 5/00; A61B 5/1455; A61B 5/28; A61B 5/318; A61B 5/01; A61B 7/04

(52) U.S. Cl. CPC A61B 5/02055; A61B 5/002; A61B 5/0022; A61B 5/14551; A61B 5/28; A61B 5/318; A61B 5/01; A61B 7/04; A61B 5/681; A61B 2560/0406; A61B 2560/0431; A61B 2560/045; A61B 2560/0468; A61B 2562/0204; A61B 2562/0271; A61B 2562/164; A61B 2562/222; A61B 2562/225; A61B 5/742; A61B 5/08

(57) ABSTRACT

A medical monitoring and reporting device includes a main body having a display screen, a microphone, a speaker, a camera, a sensor suite, and a system controller. A plurality of sensors is positioned on the bottom surface of the main body to make contact with the skin of a patient wearing the device on their wrist. The plurality of sensors and the sensor suite capture patient information including as Pulse Rate, Sp02, Blood Glucose, Cholesterol, Respiration Rate, Hemoglobin, Blood Pressure, Body Temperature, and/or Lactic Acid⋅Uric⋅HBA1c. A system memory that stores the captured patient information, and a wireless communication unit sends the patient information to an external device and permits telemedicine visits using the onboard display, microphone and speaker. External sensors are selectively connected to the device for capturing additional patient information for storage on the system memory and transmission to an external device.

### CROSS-REFERENCE TO RELATED APPLICATIONS

This application claims the benefit of U.S. application Ser. No. 17/128,574 filed Dec. 21, 2020, U.S. Application Ser. No. 63/419,483 filed on Oct. 26, 2022, and U.S. Application Ser. No. 63/545,321 filed on Oct. 23, 2023, the contents of which are incorporated herein by reference.

### TECHNICAL FIELD

The present invention relates generally to medical devices, and more particularly to a medical monitoring device.

### BACKGROUND

The statements in this section merely provide background information related to the present disclosure and may not constitute prior art.

When a patient is admitted to the hospital for routine or emergency care, they are examined by medical staff who utilize a plurality of different devices that detect and/or monitor specific medical information. Several nonlimiting examples of such devices include blood pressure monitoring devices, temperature recording devices, EKG machines, blood sugar monitoring devices, and more.

Outside of a hospital setting, users have little to no means for detecting and monitoring health information unless they have a known problem (e.g., diabetes, fever, etc.). To this end, asymptomatic issues may go unnoticed and untreated for long periods of time before symptoms arise that lead the patient to seek medical care. In some situations, this delay can result in a worsening of the underlying condition or may branch out to create additional medical issues that are caused by the underlying condition. In either instance, patients will always benefit from early detection and treatment of medical issues.

Accordingly, it would be beneficial to provide an unobtrusive medical monitoring and reporting device that can be worn continuously by a user to monitor and report patient information to both a user and a remote monitoring service.

### SUMMARY OF THE INVENTION

The present invention is directed to a medical monitoring and reporting device. One embodiment of the present invention can include a main body having a display screen, a microphone, a speaker, a camera, a sensor suite, and a system controller. A pair of wristband straps are provided to secure the main body to the wrist of a patient.

In one embodiment, a plurality of sensors is positioned along the bottom surface of the main body to make contact with the skin of a patient wearing the device at a location above the radial artery of the patient. Each of the plurality of sensors and the sensor suite work alone or together to capture patient information such as Pulse Rate, Sp02, Blood Glucose, Cholesterol, Respiration Rate, Hemoglobin, Blood Pressure, Body Temperature, and/or Lactic Acid⋅Uric⋅HBA1c.

In one embodiment, the system controller includes a memory that stores the patient information, and a wireless communication unit that sends the patient information to an external device. In one embodiment, the wireless communication unit sends and receives audiovisual information concurrently with the patient information to permit a patient to conduct a telemedicine visit.

In one embodiment, a plurality of external sensors is selectively connected to the device. Each of the external sensors capturing additional patient information for storage on the system memory and display on the display screen. The plurality of external sensors can be controlled by the system controller and the additional patient information can be transmitted to an external device via the wireless communication unit.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1A is a front view of a medical monitoring and reporting device that is useful for understanding the inventive concepts disclosed herein.

FIG. 1B is a back view of the medical monitoring and reporting device in accordance with one embodiment of the invention.

FIG. 2 is a simplified block diagram of the sensor suite of the medical monitoring and reporting device, in accordance with one embodiment of the invention.

FIG. 3 is a simplified block diagram of the system controller of the medical monitoring and reporting device, in accordance with one embodiment of the invention.

FIG. 4 is a perspective view of the medical monitoring and reporting device in operation, in accordance with one embodiment of the invention.

FIG. 5 is another perspective view of the medical monitoring and reporting device in operation, in accordance with one embodiment of the invention.

FIG. 6 is yet another perspective view of the medical monitoring and reporting device in operation, in accordance with one embodiment of the invention.

FIG. 7 is yet another perspective view of the medical monitoring and reporting device in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, the term “unit” and “module” are used interchangeably to describe a series of identified physical components which are linked together and/or function together to perform a specified function.

As described herein, the term “patient information” shall include any type of data that is captured, recorded, or collected from the sensor suite and/or external sensors of the device. Several nonlimiting examples of patient information includes, but is not limited to: Pulse Rate, Sp02, Blood Glucose, Cholesterol, Respiration Rate, Hemoglobin, Blood Pressure, Body Temperature, and/or Lactic Acid⋅Uric⋅HBA1c, among others, for example.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “connector” includes any number of different elements that work alone or together to repeatedly join two items together in a nonpermanent manner. Several nonlimiting examples of connectors include but are not limited to push-to-connect type devices, opposing strips of hook and loop material (e.g., Velcro®), attractively oriented magnetic elements or magnetic and metallic elements, buckles snaps and buttons, for example. Each illustrated connector can be permanently secured to the illustrated portion of the device via a permanent sealer such as glue, adhesive tape, or stitching, for example.

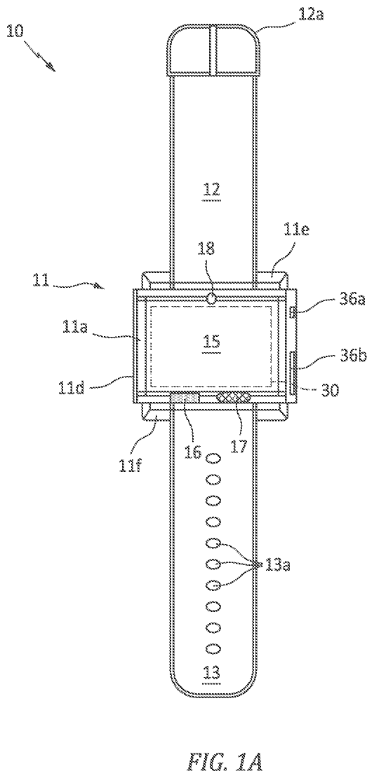

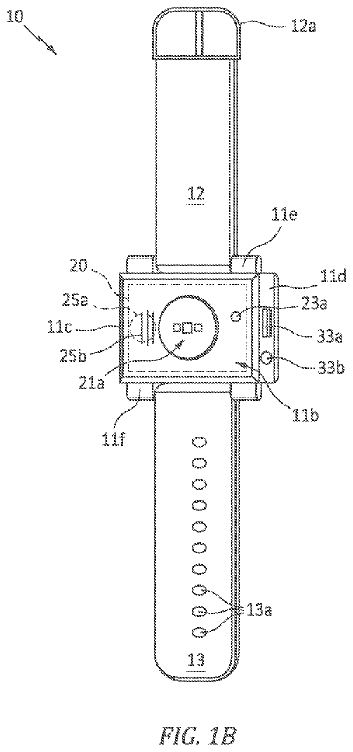

FIGS. 1-7 illustrate one embodiment of a medical monitoring and reporting device 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As shown throughout the drawings, the device 10 can include, essentially, a main body 11 having an onboard sensor suite 20 and a controller 30.

In the preferred embodiment, the main body of the device will be constructed so as to be worn on the wrist of a user and will resemble a wristwatch. Such a feature is important so as to allow the device to be generally unobtrusive, and to permit a user to comfortably wear the device at all times. By maintaining a low-profile unobtrusive design, users wearing the device will not advertise that they are experiencing or are monitoring potential medical issues, so as to not attract the attention of others with whom the user interacts.

As shown at FIGS. 1A and 1B, the main body 11 can include a central body member having a top surface 11a, a bottom surface 11b, and a plurality of side walls 11c, 11d, 11e and 11f that form a generally hollow and waterproof interior space.

The main body can include any number of different shapes and sizes and can be constructed from any number of materials that are, for example, relatively strong and stiff for their weight. Several nonlimiting examples include but are not limited to various metals or metal alloys (e.g., aluminum, steel, titanium, or alloys thereof), plastic/polymers (e.g., high-density polyethylene (HDPE), rigid polyvinyl chloride (PVC), malleable polyethylene terephthalate (PET)), and/or various composite materials.

A pair of elongated wrist bands 12 and 13 can extend outward from two side walls 11e and 11f. The wrist bands may be constructed from any number of different materials such as leather, rubber and/or various metals (e.g., gold, gold plated, aluminum, steel, etc.) Each of the wrist bands can be selectively joined together via band connectors such as the illustrated pins 12a and buckle 13a to allow a user to ensure the below described sensors are securely contacted with their skin.

Although described above with regard to two separate wrist bands that are joined together, any number of other types of connectors are also contemplated. Likewise, the wristband may be constructed as a single band having a stretchable main body or centrally located latch.

In one embodiment, the top surface 11a of the device can include, comprise, or consist of a touch sensitive display screen 15 such as an OLED display, for example, that allows two-way communication with a user. The display screen can be connected to the sensor suite 20 and controller 30 and can provide a menu of system actions that a user can select. Several nonlimiting examples include but are not limited to activating individual sensors of the sensor suite and/or the external sensors, retrieving or storing patient information, sending information to a remote monitoring station, displaying date and time information, among others, for example.

In one embodiment, the device 10 can also function to send and receive voice, data, and video information with and from any number of external devices. As such, one embodiment of the device 10 can include a microphone 16, a speaker 17 and a camera 18. Each of these components being connected to the below described controller 30.

In one embodiment, a plurality of sensors can be positioned along or within the main body 11. Each of these sensors are referred to collectively as a sensor suite 20 and can function to capture any type of patient information. The sensor suite 20 can be connected to the controller 30, so as to receive operating instructions therefrom and to allow the information captured by the sensor suite to be stored in the system memory and/or transmitted to a remote device such as a remote monitoring service, for example.

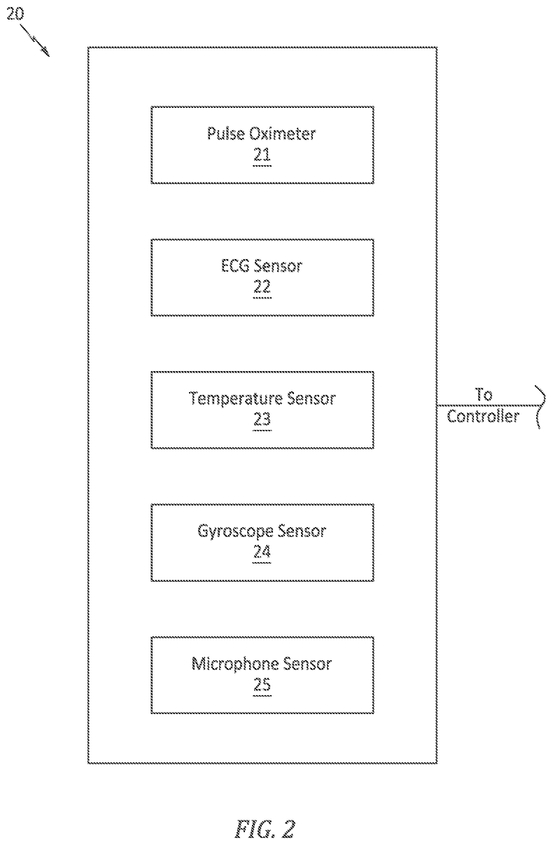

As shown at FIG. 2, one embodiment of a sensor suite 20 for use herein can include a pulse oximeter 21, an ECG module 22, a temperature sensor 23, a gyroscope 24, and a microphone 25, among others.

The pulse oximeter 21 can be communicatively linked to the below described controller 30 and can include any number of different components capable of detecting and measuring the amount of oxygen in the blood of a patient's body. In one embodiment, the pulse oximeter can include, or can be connected to a plurality of Light Emitting Diodes (LEDs) and a photodiode 21a that are positioned along the bottom surface 11b of the main body. In operation, the LED's can generate and direct light at different wavelengths, such as 660 nm and 910 nm, for example through the radial artery on the wrist of the patient wearing the device. This light can be detected by the photodiode and calculated to determine blood oxygen levels which can be shown in real time on the display screen 15 and stored in the device memory.

In addition to the above, the memory can be encoded with instructions such as various algorithms and formulations for allowing the processor to determine patient information from the raw data captured by the pulse oxygen sensor and/or the LED's and photodiode. Several nonlimiting examples of additional patient information can include the patients: Pulse Rate, Sp02, Blood Glucose level, Cholesterol, Respiration Rate, Hemoglobin, and/or Lactic Acid⋅Uric⋅HBA1c, for example.

One example of a suitable pulse oximeter includes the I2C based Max32664 Biohub sensor that is commercially available from SparkFun Electronics, inc. Of course, this is but one possible arrangement of components for use in detecting and determining the pulse oxygen level of a patient using the device 10. To this end, any number of other components and arrangement of components are contemplated for use herein.

The ECG sensor 22 can be communicatively linked to the below described controller 30 and can include any number of different components capable of detecting and calculating the electrical movement of the heart. In one embodiment, the ECG sensor can utilize the above-described Light Emitting Diodes (LEDs) and a photodiode 21a or can have a second dedicated LED and photodiode to capture information from the radial artery on the wrist. This information can be processed through the ECG module to generate a single lead ECG pattern of the patient in real time on the display screen 15 and for storage by the device memory.

One example of a suitable ECG sensor includes the AD8232-ECG-sensor that is commercially available from SparkFun Electronics, Inc. Of course, this is but one possible arrangement of components for use in detecting and determining the pulse oxygen level of a patient using the device 10. To this end, any number of other components and arrangement of components are contemplated for use herein. In this regard, the ECG module 22 can be communicatively linked—either directly or via the controller 30—to the below described communication port 33b so as to communicate with a remote sensor such as the remote ECG sensor 70. Such a feature allowing the device 10 to perform a complete and thorough multi-lead ECG reading of a patient wearing the device.

The temperature sensor 23 be communicatively linked to the below described controller 30 and can include a thermocouple 23a that is positioned along the bottom surface 11b of the main body so as to make direct contact with the skin of a patient wearing the device. The temperature sensor can function to detect and provide the temperature of the patient wearing the device 10 to the display screen 15 and for storage in the device memory.

One example of a suitable temperature sensor includes the DS-18B20 temperature sensor that is commercially available from ALLECIN, Inc. Of course, this is but one possible arrangement of components for use in detecting and determining the temperature of a patient using the device 10. To this end, any number of other components and arrangement of components are contemplated for use herein.

The gyroscope sensor 24 can be communicatively linked to the below described controller 30 and can function to detect and measure movement of the device. In the preferred embodiment, the gyroscope sensor will be at least a 3-axis sensor capable of detecting rotational velocity and rate of change of angular position over time. In operation, the gyroscope sensor can function primarily to detect impacts that would be associated with a user falling or being involved in a car accident.

As will be described below, the controller can display a message to the user upon detecting movement associated with such incidents, and if the message is not responded to and/or no additional movement is detected within a predetermined period of time (e.g., 30 seconds), the system can notify emergency services.

One example of a suitable gyroscope sensor includes the MPU-6050 Gyroscope sensor that is commercially available from HiLetgo Inc. Of course, this is but one possible arrangement of components for use in detecting and determining movements of a patient using the device 10. To this end, any number of other components and arrangement of components are contemplated for use herein.

The microphone sensor 25 can be communicatively linked to the below described controller 30 and can include any type of listening device that can function to detect and capture sounds. In one embodiment, a digital microphone 25a can be positioned above a series of openings 25b that are located along the bottom surface of the main body 11b. The openings using the thickness of the main body material to provide a physical separation distance and an unimpeded path between the microphone's input and a patient's body so as to allow the microphone to capture sounds from the patient such as the sound of blood flowing through the radial artery on the wrist of the patient wearing the device, the sound of the patients' heart.

beat, and/or lungs, for example.

The patient information from the microphone can be stored within the device memory, played audibly via the main body speaker, and/or transmitted to an external device. One example of a suitable microphone sensor includes the INMP441 high performance microphone that is commercially available from TDK InvenSense Inc. Of course, this is but one possible arrangement of components for use in detecting and listening to sounds from the body of a patient using the device 10. To this end, any number of other components and arrangement of components are contemplated for use herein.

The sensor suite 20 can be coupled to the below described controller 30, in order to receive operating instructions, and to allow the patient information gathered by the sensors to be stored and/or transmitted to an external device such as a remote monitoring service, for example, in real time. Although described above as including specific sensors and sensor components, this is for illustrative purposes only, as those of skill in the art will recognize that any number of additional sensors and/or sensor components can be provided in order to capture, store and send any type of patient information.

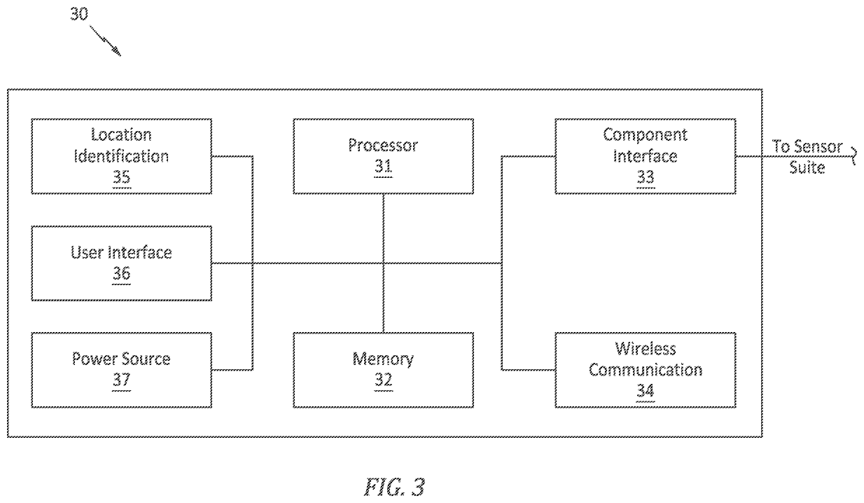

FIG. 3 is a simplistic block diagram illustrating one embodiment of the system controller 30. The controller can be connected to and control an operation of the sensor suite 20 and each of the plurality of external sensors 50, 60 and 70 described below. Additionally, the system controller can communicate wirelessly with any number of externally located computers, smartphones and/or processor enabled devices such as a healthcare office or remote monitoring service, for example.

One embodiment of the system controller 30 can include a processing unit 31 that is conventionally connected to an internal memory 32, a component interface unit 33, a wireless communication unit 34, a location identification unit 35, a user interface 36, and/or a power unit 37.

Although illustrated as separate elements, those of skill in the art will recognize that one or more system components 31-37 may include, comprise, or consist of one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a solid-state hard drive, for example.

In various embodiments, the memory can contain any number of different programs that permit the processor to perform the functionality described herein, such as controlling the operation of each element of the sensor suite 20, and the external sensors via the component interface, for example. The memory device can also function to receive and store patient information from the sensors.

The component interface unit 33 can function to provide a communicative link between the processing unit 31 and various system elements such as camera 18, speaker 17, microphone 16, display 15, the individual sensors of the sensor suite 20, the communication unit 34, and the power unit 36, among others, for example. In this regard, the component interface unit can include any number of different components such as one or more PIC microcontrollers, standard bus, internal bus, connection cables, and/or associated hardware capable of linking the various components.

In one embodiment, the component interface unit can include, or can be connected to one or more cable receptacles/plugs such as a USB port 33a, an RCA plug 33b, computer ports, or other such receptacles that are capable of engaging a communication cable that is connected to one or more of the below described external sensors. Of course, any other means for providing the two-way communication between the system components can also be utilized herein.

The wireless communication unit 34 can include any number of components capable of sending and/or receiving electronic signals with another device, either directly or over a network. In one preferred embodiment, the communication unit 34 can include a local transceiver such as a Bluetooth or Wi-Fi transceiver, for example. In this regard, the wireless communication unit can allow the device 10 to receive operating instructions and to send patient information with other devices.

Of course, the communication unit is not limited to the use of these particular components, as any number of other transmission and reception mechanisms and protocols can also be utilized herein. Several nonlimiting examples include cellular transceivers using a cellular communications network, satellite transceivers using a satellite network, Low-Energy Bluetooth (BLE), Near-Field-Communication (NFC) devices, UHF, and/or radio transmitters and receivers, and/or Long-Range Wide Area Networks, for example.

The location identification unit 35 can function to provide real time location information (e.g., address, GPS coordinates, etc.) of the device 10 at all times. In one embodiment, the location unit can include a discrete GPS signal antenna, and transceiver for communicating with any number of third-party location tracking companies that provides tracking and/or location services for registered GPS enabled devices. Alternatively, or in addition thereto, the location unit can utilize the cellular transceiver of the device to interact with the mapping and location services offered by the cellular provider and/or the Wi-Fi router connection to identify the approximate location of the device.

In either instance, the captured location information can be determined and stored by the memory and can be selectively sent to any number of other devices. For example, the location information can be included in transmissions of patient information to a remote monitoring company and/or can be sent to an emergency contact or first responders (e.g., police or ambulance) in an emergency situation wherein the gyroscope sensor detects a user has fallen or been involved in a car accident, for example.

The user interface 36 can function to accept user inputs and/or to provide information to a device user. In various embodiments, the user interface can include or control the above-described display screen 15 and any number of buttons/switches 36a and 36b, that are connected to the processor 31 so as to activate various programmatic functions such as the operation of the sensor suite and/or external sensors, for example.

The power unit 37 can function to supply the required power to each of the system components. In one embodiment, the power unit can include one or more onboard batteries which may be recharged via the communication port 33a, for example. In one embodiment, the power unit can include a wireless charging unit to permit the device to be charged in a wireless manner.

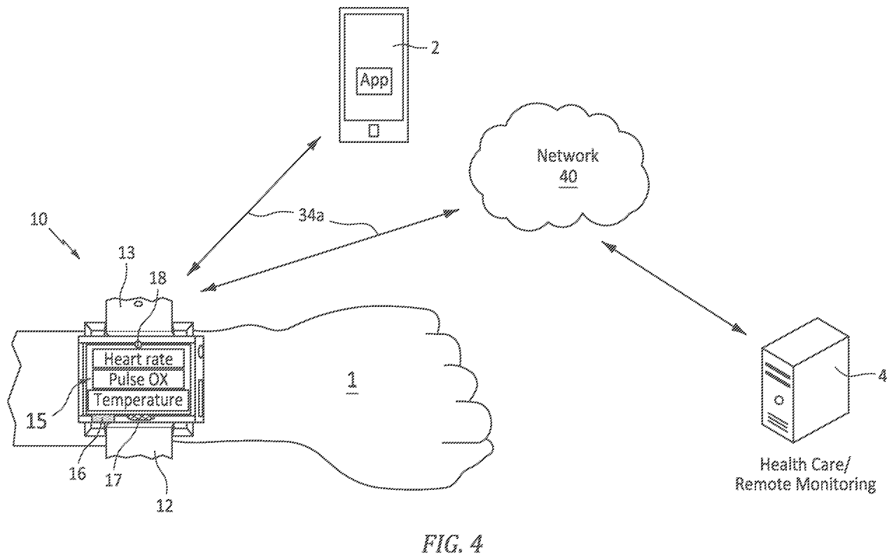

FIG. 4 illustrates one embodiment of the device 10 in operation. As shown, the straps 12 and 13 can be positioned about the wrist of a user 1 to position the bottom surface of the main body onto the skin of the user directly above the radial artery on the user's wrist. When so positioned, the user can engage the display screen 15 to selectively activate the sensor suite to capture and/or display patient information.

In various embodiments, the patient can selectively send some or all of the patient information stored in the device memory or that is being captured live by the sensors to an external device. For example, the device 10 can communicate directly with a user's smartphone 2 or can communicate over a network 40 such as the internet, for example, with a computer 4 at a hospital or healthcare providers office 4. Such a feature allows the healthcare provider office to continuously and remotely monitor the vitals of the patient, and in the event the patient information presents a concern (e.g., high blood pressure, imminent heart attack, low or high glucose level) the service 4 can contact the patient.

In this regard, the device 10 can also function to receive voice, data, and video information from any number of external devices. As such, a user can employ one or more of the microphone 16, speaker 17 and camera 18 to conduct a telemedicine visit with a health care provider/remote monitoring service 4 or other such individual, wherein the sensor suite provides patient data to one or both of the patient or the health care provider in real time concurrently with the voice and video teleconference.

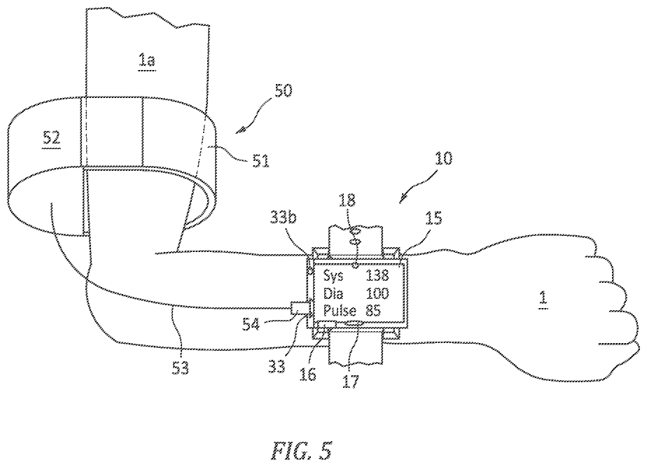

As noted above, the device 10 can function to engage and receive data from any number of externally located sensors capable of individually capturing patient information. To this end, FIG. 5 illustrates one embodiment of an external blood pressure sensor 50 that is connected to the device 10 to provide a secondary blood pressure reading of the user's upper arm 1a.

As shown, the cuff sensor 50 can include an inflatable cuff 51, a battery powered pump, manometer and CPU controller 52, that are connected to an elongated cable 53 having a plug 54 such a USB plug, for example along one end. In this regard, the device user can position the cuff about their upper arm, connect the plug 54 to the communication port 33a and utilize the display screen 15 to inflate the cuff and receive the patient data from the cuff in the form of the detected blood pressure.

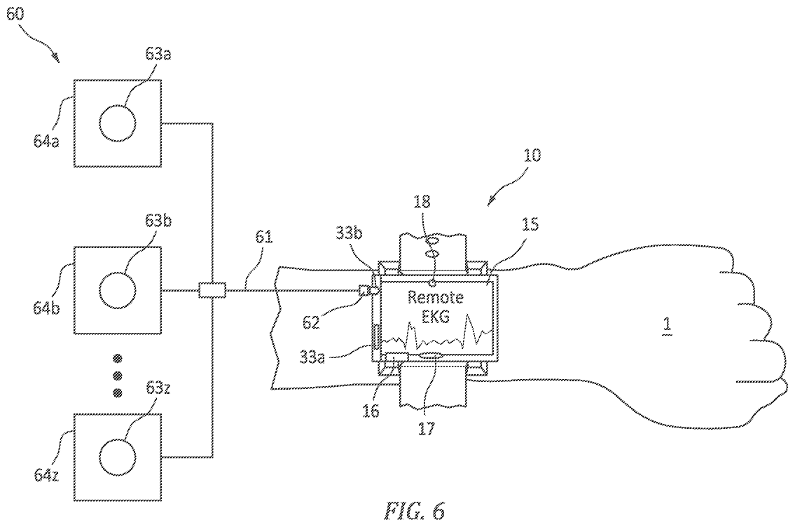

FIG. 6 illustrates one another embodiment of a remote sensor in the form of an ECG sensor 60. As shown, the remote ECG sensor 60 can include an elongated cable 61 having a plug 62 such as an RCA plug along one end that is connected to one of the communication ports 33a of the device 10. A plurality of electrodes 63a-63z are positioned along the second end of the cables and can be connected to disposable or reusable adhesive buttons 64a-64b. The electrodes and buttons can be selectively positioned along the chest and/or abdominal area of the patient to provide a complete and multi-view picture of the electrical activity of the patient's heart. The remote ECG sensor 60 can be activated, powered and controlled by the device 10 to capture and report patient information thereto.

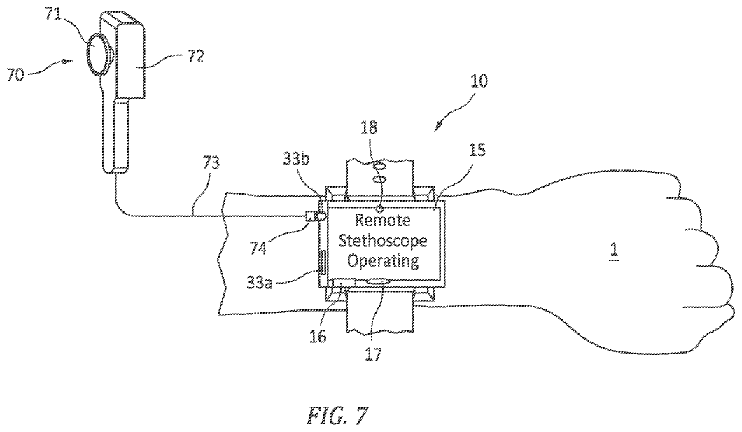

FIG. 7 illustrates one another embodiment of a remote sensor in the form of a digital stethoscope 70. As shown, the stethoscope 70 can include a digital microphone 71 that is positioned within a housing 72. The digital microphone is connected to an elongated cable 73 having a plug 74 such as an RCA plug, for example, that engages the communication port 33a of the device 10. The user can selectively position the stethoscope over different areas of their body such as their heart, lungs or bowels, for example, to permit the microphone to detect and capture internal sounds. The remote digital stethoscope 70 can be activated, powered and controlled by the device 10 to capture and report patient information thereto.

Although described and illustrated with regard to particular remote sensors this is for illustrative purposes only. To this end, any number of different sensors can be removably connected to the device 10 to capture any type of patient information. The captured information from each remote sensor can be displayed on the device screen 15, played by the device speaker 17, stored by the device memory and/or selectively transmitted to any number of external devices in the same way information from the sensor suite is.

As to a further description of the manner and use of the present invention, the same should be apparent from the above description. Accordingly, no further discussion relating to the manner of usage and operation will be provided.

As described herein, one or more elements of the device 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A device, comprising:
a main body having a plurality of walls that define an interior space;
a display that is positioned along a top surface of the main body;
a plurality of sensors that are positioned along one of the plurality of walls of the main body;
a sensor suite that is positioned within the main body, said sensor suite being connected to each of the plurality of sensors; and
a controller that is connected to each of the display and the sensor suite,
wherein the plurality of sensors and the sensor suite are configured to capture patient information of a patient.

2. The device of claim 1, further comprising:
a wristband that is connected to the main body, said wristband being configured to secure the main body to a wrist of the patient.

3. The device of claim 2, wherein the plurality of sensors are positioned along a bottom wall of the main body and are configured to physically touch the wrist of the patient.

4. The device of claim 1, wherein the plurality of sensors includes a plurality of light emitting diodes and a photodiode.

5. The device of claim 4, wherein the sensor suite includes a pulse oximeter sensor that is connected to the plurality of light emitting diodes and the photodiode.

6. The device of claim 5, wherein the patient information includes a pulse oxygen level of the patient.

7. The device of claim 4, wherein the sensor suite includes an ECG sensor that is connected to the plurality of light emitting diodes and the photodiode.

8. The device of claim 7, wherein the patient information includes a single lead ECG reading of the patient.

9. The device of claim 1, wherein the plurality of sensors includes a thermocouple.

10. The device of claim 9, wherein the sensor suite includes a temperature sensor that is connected to the thermocouple.

11. The device of claim 10, wherein the patient information includes a body temperature of the patient.

12. The device of claim 1, wherein the plurality of sensors includes a digital microphone.

13. The device of claim 12, wherein the sensor suite includes a microphone sensor that is connected to the digital microphone.

14. The device of claim 13, wherein the patient information includes sounds from an inside portion of a body of the patient.

15. The device of claim 1, wherein the controller comprises:
a system memory;
a communication unit; and
a processor that is connected to each of the system memory and the communication unit.

16. The device of claim 15, wherein the communication unit includes functionality for sending the patient information to an external device.
